(19) United States
(12) Patent Application Publication
(10) Pub. No.: US 2013/0184247 A1
(43) Pub. Date: Jul. 18, 2013

(54) METHOD FOR PREPARATION OF NICOTINIC ACID COPPER CHLORIDE COMPLEX, AND ITS PHARMACEUTICAL USES

(71) Applicant: Salah Fathiy Hussein Aly Ibraheim, Cairo (EG)

(72) Inventor: Salah Fathiy Hussein Aly Ibraheim, Cairo (EG)

(21) Appl. No.: 13/786,275

(22) Filed: Mar. 5, 2013

Related U.S. Application Data
(63) Continuation of application No. 13/016,369, filed on Jan. 28, 2011, now abandoned.

(30) Foreign Application Priority Data
Sep. 21, 2008 (EG) PCT/EG2008/000034

(51) Int. Cl. C07D 213/79; A61K 45/00; A61K 31/555

(52) U.S. Cl. CPC C07D 213/79; A61K 31/555; A61K 45/00
USPC 514/188; 546/8

(57) ABSTRACT

A method for preparation pharmaceutical compounds in large quantities consists of copper (I) halide, preferably the chloride complex of nicotinic acid characterized by elemental analysis, spectroscopy and crystallographic methods, used for treatment of muscular dystrophy, myopathy, myasthenia gravis, parkinsonism, Chronic Fatigue Syndrome, Male Infertility and post stroke muscle weakness.

Chlorobis (nicotinic acid) copper (I) monohydrate complex, its composition [CuCl(nicotinic acid)₂]. H₂O, has been prepared and its physical and chemical properties as well as its crystal structure have been investigated for the first time.

### CLAIM OF PRIORITY

This is a continuation of copending U.S. application Ser. No. 13/016,369, filed on Jan. 28, 2011, which was a continuation of and claims the priority of copending U.S. application Ser. No. 13/055,428, filed on Jan. 21, 2011, and of PCT/EG2008/000034 and of its Egyptian priority document, including all foreign filing applications.

### TECHNICAL FIELD

Drugs, Pharmaceuticals

### BACKGROUND

Duchene Muscular Dystrophy (DD) is an X linked disorder primarily affecting skeletal muscles. It is caused by the lack of dystrophin, the protein product of DD gene, located on XP21chromosome. The patients are males who suffer from progressive muscle weakness extending to both cardiac and respiratory muscle failure. Patients suffering from DD, die from respiratory and/or cardiac failures at an age of 25-30 years. Female siblings are healthy carriers.

Male infertility is a multi factorial disease process with a number of potential contributing causes. Considering that the majority of male infertility cases are due to deficient sperm production of unknown origin, environmental and mutational factors must be evaluated.

The treatment of male factor infertility is a rapidly developing field. Introduction of microsurgical fertilization techniques allows assisted conception units to treat couples who previously would not have benefited from in vitro fertilization techniques.

However, these techniques are only used for the minority of sub-fertile men in andrologial practice. Many sub-fertile men are still treated pharmacologically or by sperm selection methods to enhance sperm fertilizing ability. Numerous pharmacological compounds have been described that enhance sperm motility and thus, potentially sperm fertilizing capability. Sperm motility plays an important role in the normal fertilization process. Poor sperm motility (<50% motile sperm with <2+ forward progression according to WHO protocol), is considered a major factor in diminished rates of fertilization. Medical trials for male non obstructive infertility by hormonal replacement, corticosteroids (in immune infertility). Mutational therapies were carried out but, results were not satisfactory.

Chronic fatigue is a world wide complaint affecting the productivity of a good percentage of the population. Chronic fatigue includes: unexplained fatigue, chronic fatigue syndrome, and fibromyalgia. In all types of fatigue, the complex relieved muscle pain increased the physical activity and productivity and relieved associated depression of the patients.

### RELATIONSHIP OF THE INVENTION TO THE PRIOR ART

A different copper complex for pharmaceutical use was disclosed in published application PCT EGY 116/030/2006.

A Copper Nicotinate complex was an active ingredient in (Royal Top)® a food supplement preparation previously sold only in Egypt (registration No. 97/1740); but which was withdrawn from the market by the Egyptian Ministry of Health due to the following evident drawbacks, not met with the present invention:

The formulation procedure contains addition of water which is contraindicated for the stability of copper (I).

The Iron oxide included in the above named formula affords high probability of displacement of copper from the active ingredient.

Parabenzoates which were needed as preservatives are undesirably for human consumption.

Preparation of the copper (I) nicotinate complex according to the procedure by, M. A. S. Gohar and M. Dratovsky, Collection Czechoslov. Chem. Commun. 40, 26 (1975), did not give reproducible results coping with the declared analyses of the unhydrated formula of the complex (the structure referred by authors in the reference).

HPLC analyses of the complex prepared according to the procedure published by Gohar and Dratovsky, revealed a heterogeneous sample.

### GENERAL STATEMENT OF THE INVENTION

The entire disclosure of U.S. application Ser. No. 13/055,428, including the specification, claim, abstract and drawings, is incorporated herein by reference, as if fully repeated herein in haec verba.

Copper (I) chloride complex of nicotinic acid was prepared, characterized by elemental analysis, IR, UV-visible spectra, and its crystal structure was determined by single crystal diffraction method. As a drug in a pharmaceutically acceptable composition, this compound exerts a very positive influence on different incurable diseases, e.g. muscular dystrophy, myopathy, myasthenia gravis, parkinsonism, Chronic Fatigue Syndrome, Male Infertility and post stroke muscle weakness are examples.

The compound of the invention is a copper chloride complex with a ligand containing nicotinate residue, different from the material of the prior art: M. A. S. Gohar and M. Dratovsky, Collection Czechoslov. Chem. Commun. 40, 26 (1975), and M. A. S. Gohar and T. C. W. Mak, Polyhedron 14(17-18),2587(1995). The complex was prepared by a procedure essentially modified with respect to that published by M. A. S. Gohar et al.

### MODES OF CARRYING OUT THE INVENTION

An effective amount of a copper (I) nicotinate complex, to achieve a desired level for ameliorating fatigue, infertility, weakness of muscles, etc., is administered orally to a human. The composition for this purpose is presented as capsules, tablets, etc. The specific dose level for a particular person depends on a variety of factors including age, general health, sex, diet, body weight, and the time of administration.

According to another broad form of the invention, a method comprising the administration to a human, of an effective amount of the copper (I) nicotinate complex of this invention in a pharmaceutically acceptable formulation with other cell building factors, carriers, diluents and or excipients. The concentration of the copper (I) nicotinate complex and other adjuvant depends on different factors, e.g. type and reason of weakness of the muscles, age of the patient, etc.

According to another broad form of the invention there is provided a method for treatment of Fatigue. The method comprises administering to a human the copper (I) nicotinate complex and /or a pharmaceutically acceptable composition of the complex. The concentration of the copper (I) nicotinate complex in the pharmaceutically acceptable form is variable and depends on several factors, e.g. age of the patient, reason of fatigue, etc. According to a further broad form of the invention there is provided a method for treatment of infertility in men.

The copper (I) nicotinate complex of invention can be used as such or can be formulated in combination with other medicaments, e.g. a non-steroidal, anti-inflammatory, I. Monech, H. Prentice, Z. Rickaway, and H. Weissbach, PNAS 106(46), 19611-19616(2009)].

Such dosage forms may also comprise, as normal practice, additional substances other than inert diluents, such as food supplements:, e.g., L-carnitine, L-arginine, Nacetylcysteine, resviratrol, vitamin B₆, vitamin B₁, nicotinamide, folic acid, α-tocoferol, lemon grass, ginseng, omega-3, lipoic acid, wheat germ oil, ascorbic acid, other water soluble vitamins, etc. In the case of capsules the dosage forms may also comprise buffering agents.

Solid dosage forms for oral administration may include capsules, tablets, pills and granules, in such solid dosage forms, the copper (I) nicotinate complex complex may be admixed with at least one inert diluent. The dose can vary according to age, the root of administration and the state of the patient in relation to the illness.

The effective doses vary within orders of magnitude from 0.1 mg/Kg to 0.75 mg /Kg body weight of the patient.

### BRIEF DESCRIPTION OF THE DRAWINGS

FIG. 1: RP-HPLC analysis of the complex prepared according to previous work

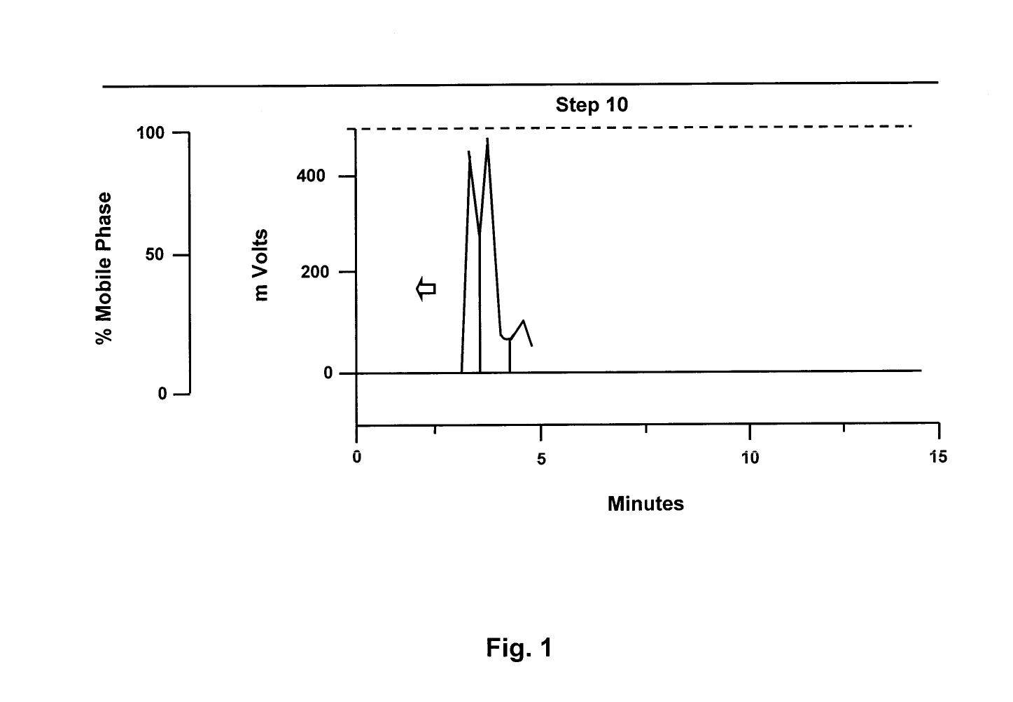

Complex prepared according to previous work [M. A. S. Gohar and M. Dratovsky, Collection Czechoslov. Chem. Commun. 40, 26 (1975); M. A. S. Gohar and T. C. W. Mak, Polyhedron 14(17-18),2587(1995] gave 3 peaks with RP-HPLC analysis indicating a heterogeneous product.

The data for the first two peaks are shown in this Table 1. The full graph is shown in FIG. 1, showing three peaks.

FIG. 2: RP-HPLC analysis of the complex prepared according to our invention, from the procedures of Examples 1-4:

Homogenous copper (I) nicotinate complex of invention by RP-HPLC analysis. Reversed phase—High performance liquid chromatography (RP-HPLC) of the copper (I) nicotinate complex of invention revealed a single peak at Rt 3.06 min.
Column: C18
Mobile phase: Methanol/Acetonitrate
UV detector: 240 nm
Volume injected: 20 μL
   Model of apparatus: Gilson

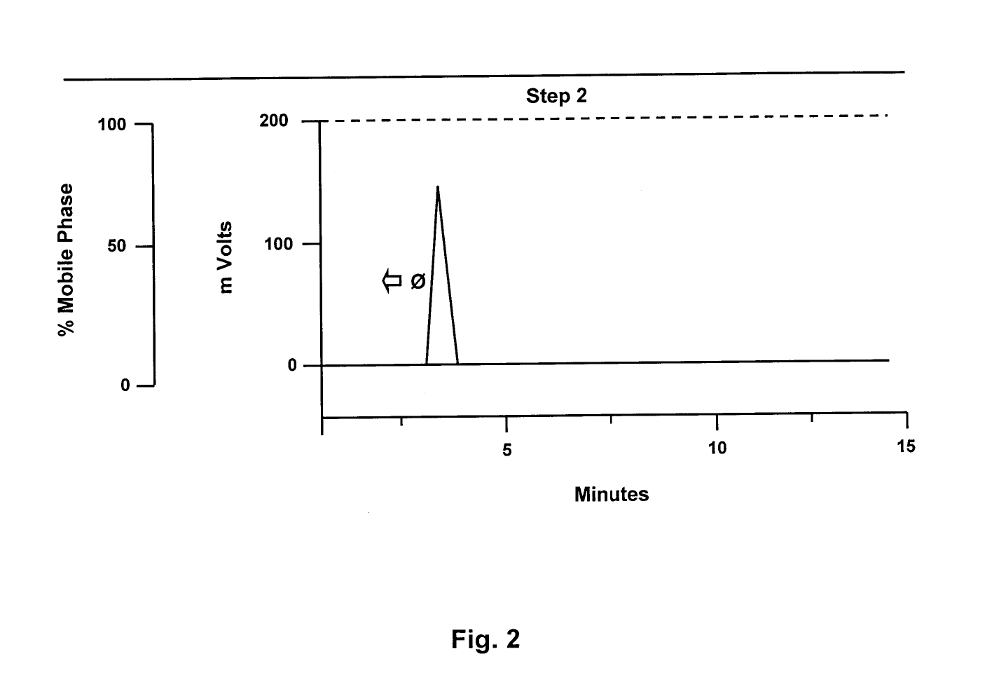

The datum point for the single peak is shown in this Table 2. The graph is shown in FIG. 2.

### DETAILED DESCRIPTION OF THE INVENTION

In order that the present invention can be more readily understood, reference is now made to the following examples.

### Example 1

### (A)—Preparation of Copper (I) Chloride

Procedure 1: Solution of purified Copper (II) chloride (40.2 g, 0.3 mol) in (100 ml) water was added gradually with efficient stirring to a solution of Lascorbic acid (26.5 g, 0.15 mol) in (130 ml) water. The mixture was left to stand; the supernatant must be free of color. Precipitated Cu(I) Cl was filtered by suction and washed with absolute ethanol. The product was then dried at 50-60° C. for 30-60 min. Yield 22 g (74.4%).

Procedure 2: Solution of L-ascorbic acid (35.2 g, 0.2 mol) in (150 ml) water was added gradually with efficient stirring to a solution of purified Copper (II) chloride (40.2 g, 0.3 mol) in (100 ml) water. Precipitated Cu(I) Cl was filtered and washed with 60-80% ethanol and then dried by suction. Yield 23 g (77.8%).

### Example 2

### (B)—Preparation of Nicotinic Acid Solution

Procedure 1: Pure nicotinic acid (30.75, 0.25 mol) was dissolved in 60-80% ethanol (2.5 L) by stirring and the solution was heated to boiling then Lascorbic acid (50 g) was added and stirred till dissolved.

Procedure 2: To a mixture of pure nicotinic acid (30.75, 0.25 mol) and Lascorbic acid (50 g) was added 50 ml water and 2.5 L of absolute ethanol and the mixture was stirred and heated to boiling till clear solution was obtained.

### Example 3

### C—Preparation of the Copper (I) Nicotinate Complex

Procedure 1: A freshly prepared suspension of Cu (I) Cl (9.85 g, 0.1 mol) Example 1 in 60-80% ethanol (150 ml) was added gradually to an efficiently stirred and heated solution B. The obtained turbid solution was filtered wile hot and the filtrate was left over night.

Procedure 2: A freshly prepared suspension of Cu (I) Cl (9.85 g, 0.1 mol) Example 1 in absolute ethanol (150 ml) was added gradually to an efficiently stirred and boiling solution B. The obtained turbid solution was boiled and filtered while boiling and the filtrate was left over night.

### Example 4

### D—Isolation of the Copper (I) Nicotinate Complex

Procedure 1: The reddish brown precipitate separated from the filtrate in Example 3 was filtered and washed several times with absolute ethanol and finally with acetone. The product was collected and dried at 55-60° C. for 30-60 min. Yield 9.5 g.

Procedure 2: Filter by vacuum the reddish brown precipitate separated from the filtrate Example 3, wash with aqueous ascorbic acid 5% w/v solution then with ethanol 60-90% and finally with least amount of acetone. The product was left to dry in air over night. Yield 9.2 g.

### Example 5

### E=Preparation of the Copper (I) Nicotinate Complex Ointment (20%)

Twenty grams of the copper (I) nicotinate complex was finely powdered in a whole glass mortar then Vaseline (80 g) was added portion wise and thoroughly triturated with the powder to obtain a homogenous mass. Stainless steel spatula was used to collect and to transfer the semisolid mass to a brown colored screw caped glass container.

The ointment was stored for more than two years at ambient conditions of temperature and humidity protected from direct sun light with no visual change of properties and clinical efficacy.

This preparation was applied in thin film once daily for treatment of female hair loss, skin lesions and surgical wounds.

### Example 6

### Preparation of the Copper (I) Nicotinate Complex Ointment (5%)

Five grams of the copper (I) nicotinate complex was finely powdered in a whole glass mortar then Vaseline (95 g) was added portion wise and thoroughly triturated with the powder to obtain a homogenous mass. The semisolid mass was transferred to a brown colored screw caped glass container.

The ointment was stored for more than two years at ambient conditions of temperature and humidity protected from direct sun light with no visual change of properties and clinical efficacy.

This preparation was applied in thin film once daily for treatment of burns and skin cosmetics.

### Example 7

### Recovery of Copper

Recovery of copper not consumed in the target complex formation was efficiently affected to afford on one hand environment friendly procedure through minimizing release of copper to the sewage. On the other hand more economic procedure was achieved by regeneration of copper in a suitable form to be recycled.

The residue on the filter (Example 3) was boiled with sodium hydroxide (5%) aqueous solution for 5-10 minutes and glucose (5%) solution was added in sufficient quantity and solution boiled till no more red precipitate was separated. Filter the red residue, wash with distilled water and dry in air. 3.5 g of Cu2O was recovered.

### Example 8

Oral Dosage Form:

A typical dosage form for oral administration of copper (I) nicotinate complex was prepared according to the following prescription.

Procedure 1 (binary preparation): the copper (I) nicotinate complex (10-20 mg) was mixed with L-ascorbic acid (10-50 mg) under conditions to avoid access of unwanted moisture and delivered into colored hard gelatin capsule.

This preparation was recommended for treatment of muscular dystrophy in particular Duchene and Becker MDs.

Procedure 2 (multicomponent preparation): a) the copper (I) nicotinate complex (10-20 mg) was mixed with L-ascorbic acid (10-50 mg) under conditions to avoid access of unwanted moisture (the binary preparation) and then manipulated into a microencapsulated form; b) L-argentine (500-2000 mg) was manipulated into a microencapsulated form; c) preparations a) and b) were deliver sequentially to a colored hard gelatin capsule.

This preparation was recommended for treatment of male infertility and fatigue syndrome.

### Example 9

### Skin Patches Containing Copper (I) Nicotinate Complex

The copper (I) nicotinate complex molecular weight (362.5) is suitable for skin patches formulation. One hundred milligrams were mixed with the suitable adhesive matrix and kept protected from light till used as under arm or chest application.

### Analytical Data of the Copper (I) Nicotinate Complex of Invention which is the Active Ingredient in all the Mentioned Pharmaceutical Preparations

Found %:

| | | | |
|---|---|---|---|
| C: 39.76 | H: 3.73 | N: 7.45 | Cu: 17.17 |

Calc. % for C₁₂H₁₂N₂O5ClCu, MW 362.5:

| | | | |
|---|---|---|---|
| C: 39.72 | H: 3.31 | N: 7.72 | Cu: 17.37 |

### Physical Properties

Color of the copper (I) nicotinate complex of invention:
Bright reddish-brown microcrystalline powder

Solubility of the copper (I) nicotinate complex of invention:

The complex is insoluble in non polar solvents, e.g. benzene, carbon tetrachloride, etc., and insoluble in polar solvents: water, methanol, ethanol, acetone, but soluble in these solvents upon heating in inert atmosphere, otherwise oxidation takes place.

Solubility in DMF and DMSO is advantageous when used in skin patches to carry the copper complex of this invention through the skin and into the system.

Infrared spectrum of the copper (I) nicotinate complex of invention: Exhibits C═O band around 1710 cm−1, and C—O around 1310 cm−1(KBr pellets) The composition of the complex formulated as [Cu Cl (nicotinic acid)2] was confirmed by the single crystal diffraction.
Reversed phase—High performance liquid chromatography (RP-HPLC) revealed a single peak at Rₜ 3.06 min.
Column: C18
Mobile phase: Methanol/Acetonitrate
UV detector: 240 nm
Volume injected: 20 μL
Model of apparatus: Gilson

The above examples and descriptions are intended to be exemplary only. It is understood that the full scope of this invention should be determined only by the scope of the claims set forth below.

1. The method for preparation of nicotinic acid copper chloride complex, the method for which comprises adding, drop wise, a suspension of copper (I) chloride in aqueous alcohol, to nicotinic acid dissolved in aqueous alcohol to obtain a reactant solution, from which develops a clear reddish precipitate of the nicotinic acid copper (I) chloride complex, having the empirical chemical formula:
C₁₂H₁₂ClCuN₂O₅.

2. In the method according to claim 1 the final mixture was allowed to stand over several hours until the crystalline precipitate was formed.

3. In the method according to claim 2. the precipitate was filtered utilizing a pump and washed with ascorbic acid solution in alcohol, the solution comprising 70-90% ascorbic acid, further washings with ethanol 70-99%, acetone and dried under vacuum.

4. In the method according to claim 1 where the reactant solution is subject to boiling.

5. A nicotinic acid copper (I) chloride complex prepared in accordance with the method of claim 1, having the chemical formula:
C₁₂H₁₀ClCuN₂O₄.H₂O

6. A pharmacologically active composition for the treatment of patients suffering from muscular dystrophy, the composition comprising a unit dosage amount of a nicotinic acid copper (I) chloride monohydrate complex, and a physiologically suitable carrier.

7. The pharmacologically active composition for the treatment of patients suffering from muscular dystrophy according to claim 6, comprising in addition to the physiologically suitable carrier a material selected from the group consisting of vitamins, nutrients, diluents and excipients.

8. The pharmacologically active composition for the treatment of patients suffering from muscular dystrophy according to claim 6, wherein the nutrient is selected from the group consisting of L-carnitine, L-arginine, N-acetylcysteine, resviratrol, vitamin B₆, vitamin B₁, nicotinamide, folic acid, α-tocoferol, lemon grass, ginseng, omega-3, lipoic acid, wheat germ oil.

9. A pharmacologically active composition for the treatment of patients suffering from muscular dystrophy according to claim 6, in a solid dosage form for oral administration selected from the group consisting of capsules, tablets, pills, and granules.

10. A pharmacologically active composition for the stimulation of stem cells in a patient suffering from a pathological condition, the composition comprising comprising a unit dosage amount of a nicotinic acid copper (I) chloride monohydrate complex in accordance with claim 5.

11. A pharmacologically active composition according to claim 6, for inducing mitochondrial regeneration and multiplication at the pathological site of a patient.

12. A pharmacologically active composition comprising a unit dosage amount of the nicotinic acid copper (I) chloride monohydrate complex of claim 5, intended to induce the catalytic release of nitric oxide from thionitric oxide (SNO) compounds formed endogenously in a patient.

13. A pharmacologically active composition comprising a unit dosage amount of a nicotinic acid copper (I) chloride monohydrate complex in accordance with claim 5, for the treatment of fatigue.

14. A pharmacologically active composition comprising a unit dosage amount of a nicotinic acid copper (I) chloride monohydrate complex in accordance with claim 5, in semisolid dosage form for the treatment of male infertility.

15. A pharmacologically active composition for the treatment of patients suffering from burns or scars, the composition comprising a unit dosage amount of a nicotinic acid copper (I) chloride monohydrate complex in accordance with claim 5, in a semisolid dosage form for topical skin application.

16. A pharmacologically active composition for the treatment of patients suffering from suffering from Parkinsons disease, myasthenia gravis, spermatogenesis, and muscle weakness of different etiologies, the composition comprising a unit dosage amount of a nicotinic acid copper (I) chloride monohydrate complex, in accordance with claim 5.

17. A pharmacologically active composition comprising a unit dosage amount of a nicotinic acid copper (I) chloride monohydrate complex in accordance with claim 5, for the stimulation of stem cells in a patient.
